(19) United States
(12) Patent Application Publication
(10) Pub. No.: US 2024/0016378 A1
(43) Pub. Date: Jan. 18, 2024

(54) MACHINE LEARNING TO ASSESS THE CLINICAL SIGNIFICANCE OF VITREOUS FLOATERS

(71) Applicant: Alcon Inc., Fribourg (CH)

(72) Inventors: Zsolt Bor, San Clemente, CA (US); Alireza Malek Tabrizi, Fremont, CA (US)

(21) Appl. No.: 18/351,389

(22) Filed: Jul. 12, 2023

Related U.S. Application Data
(60) Provisional application No. 63/388,901, filed on Jul. 13, 2022.

(51) Int. Cl. A61B 3/10; A61B 3/00

(52) U.S. Cl. CPC A61B 3/102; A61B 3/0025

(57) ABSTRACT

Particular embodiments disclosed herein provide a method for training a machine learning model to estimate the clinical significance of floaters in a patient's eye. One or more images, such as SLO images or en face retinal OCT images, are evaluated to identify shaded regions corresponding to floaters. The shaded regions are measured and the measurements processed using a machine learning model to obtain an estimated significance. The machine learning model is then updated according to a comparison of the estimated significance to a human-assigned clinical significance. The machine learning model may additionally or alternatively be updated by evaluating the estimated category with respect to visibility threshold data, such as one or more visibility threshold surfaces defined with respect to two or more variables.

### CROSS-REFERENCE TO RELATED APPLICATIONS

This application claims benefit of and priority to U.S. Provisional Patent Application No. 63/388,901, filed Jul. 13, 2022, the entire contents of which are incorporated by reference herein in its entirety.

### BACKGROUND

Light received by the human eye passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light. The space between the lens and the retina is occupied by a clear gel known as the vitreous.

Through various causes, floaters may be present in the vitreous. A floater is typically formed of a clump of cells, collagen fibers or other tissue and is more opaque than the surrounding vitreous. Floaters cast a shadow onto the retina that causes visual disturbance for a patient, which can be quite severe in some patients.

It would be an advancement in the art to facilitate the treatment of floaters.

### BRIEF SUMMARY

The present disclosure relates generally to a system and methods for assessing the clinical significance of vitreous floaters. In one aspect, a computing device receives one or more images of a patient's eye and identifies one or more shaded regions in the one or more images. The shaded regions are processed to obtain one or more measurements of the shaded regions. The computing device processes the one or more measurements using a machine learning model to obtain an estimated clinical significance of the floaters in the patient's eye.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1A is a schematic cross-sectional representation of an eye having a floater.

FIG. 1B is an image of a retina showing a shadow caused by a floater and measurements to characterize the shadow, in accordance with certain embodiments.

FIG. 2A is a schematic block diagram of components for characterizing the clinical significance floaters using a machine learning model, in accordance with certain embodiments.

FIG. 2B is a schematic block diagram of components for characterizing the clinical significance floaters using a machine learning model and visibility threshold data, in accordance with certain embodiments.

FIG. 3 is a process flow diagram of a method for characterizing the clinical significance of floaters using a machine learning model and visibility threshold data, in accordance with certain embodiments.

FIG. 4 illustrates an example computing device that implements, at least partly, one or more functionalities of characterizing the clinical significance of vitreous floaters, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

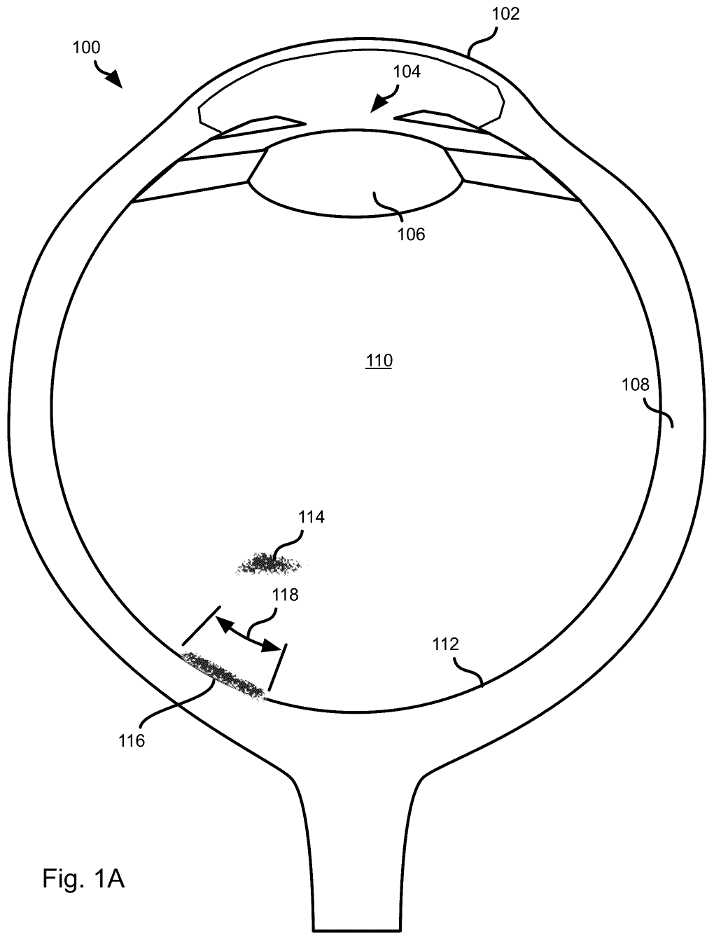

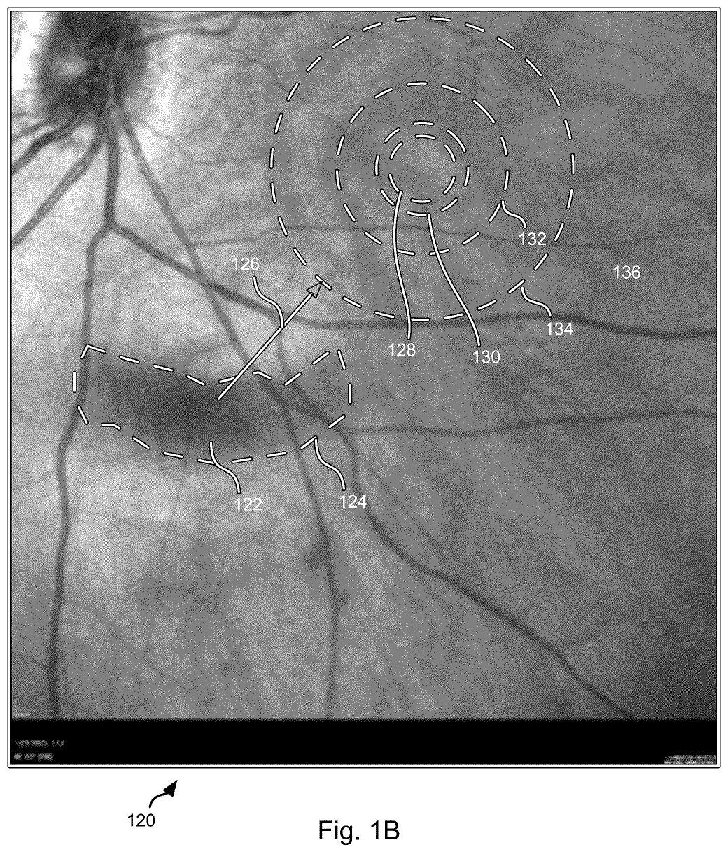

Referring to FIG. 1A, a human eye 100 includes the cornea 102, which is a sphere-like transparent layer through which light enters the eye 100. The light then passes through the anterior chamber 138, pupil 104, and lens 106 of the eye 100, respectively. The remaining volume of the globe 108 of the eye 100, known as the posterior or vitreous chamber 140, is occupied by a clear gel known as the vitreous 110. The light is focused by the cornea 102 and lens 106 onto the retina 112 at the back of the eye 100 through the vitreous 110.

Vitreous floaters 114 are clumps of cells, collagen fibers, or other contaminants within the vitreous 110. When present, a vitreous floater 114 will cast a shadow 116 on the retina 112. The shadow 116 may occupy an angular extent 118 of the field of vision of the eye 100. When sufficiently large, opaque, and/or numerous, floaters 114 can cause annoyance and significantly interfere with a patient's vision. When the shadow 116 of a floater 114 moves across the fovea, the visual acuity of the patient may be reduced.

Referring to FIG. 1B, an image 120 of the retina 112 may be obtained, such as by using a scanning laser ophthalmoscope (SLO), visible light camera, optical coherence tomography (OCT) microscope, or other imaging modality. For example, the image 120 may be an en face image captured using an SLO or OCT microscope. A portion of the light transmitted onto the retina 112 to capture the image 120 will be scattered by any floaters 114 present in the vitreous 110, thereby resulting in a shaded region 122 in the image 120.

The shaded region 122 may be identified as having contrasting pixel intensity relative to the area surrounding the shaded region 122. Floaters 114 tend to be motile such that shaded region 122 may be identified in the image 120 as having a different intensity relative to the intensity of the shaded region 122 in a prior or following image in a series of video image frames including the image 120. For example, each image in the series of images may be registered with respect to reference features of the retina, such as the pattern of vasculature (e.g., veins) of the retina in order to track and compensate for eye movement. In such an example, changes from one registered image to another may therefore correspond to shadows 116 of floaters 114. The shaded region 122 may be identified using any approach for detecting moving objects relative to a stationary background with compensation for eye movement being performed in the same manner that such approaches compensate for camera movement. Still or video images 120 may be analyzed using a machine learning model trained to identify the shaded regions 122 corresponding to floaters 114.

Various metrics of the shaded region 122 for each floater 114 may be extracted from the image 120. For example, the shaded region 122 has a size. The size may be measured as the area (e.g., number of pixels) within a boundary 124 of the shaded region. The size of the shaded region 122 may also be more simply obtained as the size of a two-dimensional bounding box that is either orthogonal (sides parallel to the rows and columns of pixels in the image 120) or oriented to conform to the shaded region 122. The size may be represented by a two-dimensional dimension of the floater 114, such as the longest line that may be contained within the shaded region 122.

The shaded region 122 may be characterized by the contrast of the shaded region 122 relative to the surrounding area of the image 120. For example, the average pixel intensity of the pixels of the image of the shaded region 122 may be divided by the average intensity of pixels outside the shaded region 122 (e.g., a band of pixels having a depth of one or more pixels around the boundary 124 of the shaded region 122) to obtain the contrast. Any approach for characterizing the contrast between regions of an image may also be used.

Movement of a shaded region 122 from one image to the next in a series of video frames 120 may also be measured or characterized. Movement may be detected using any motion-tracking approach known in the art, such as a Kalman filter or like algorithm. Movement may be characterized based on speed and/or direction of movement. For example, movement may be characterized as the component 126 of the velocity of the shaded region 122 directed toward or away from the fovea 128 (i.e., the representation of the fovea of the eye 100 in the image 120). In some implementations, the movement of the shaded region 122 is characterized as being either stationary (e.g., movement less than a threshold value), directed toward the fovea 128, or directed away from the fovea 128.

The shaded region 122 may be characterized by the location of the shaded region 122 relative to the fovea 128. For example, a distance between the shaded region 122 and the fovea 128 may be calculated as the shortest distance from the center of the fovea 128 and the point on the shaded region 122 that is closest to the center of the fovea 128. In another example, the shaded region 122 may be characterized as overlapping with the fovea 128, the perifovea 130, the parafovea 132, the macula 134, or the peripheral region 136 of the retina 112. For example, the shaded region 122 may be characterized as the innermost region of the fovea 128, perifovea 130, parafovea 132, macula 134, or peripheral region 136 that is overlapped by at least part of the shaded region 122.

The location of the fovea 128, the perifovea 130, the parafovea 132, the macula 134, or the peripheral region 136 of the eye in the image 120 may be estimated based on location in the image 120, i.e., it may be presumed that the center of the image 120 is the center of the fovea and accepted values may be used for the fovea 128, the perifovea 130, the parafovea 132, the macula 134, and the peripheral region 136. A person skilled in the art can easily recognize the fovea as a darker spot having a dimension of about 0.3 mm (about 1° visual angle) and the characteristic geometry of the retinal vasculature.

The above-described metrics of each shaded region 122 are exemplary only and other properties of each shaded region 122 may also be measured. For example, where there are multiple floaters, the metrics may include one or more separation distances between shaded regions, an average separation between floaters, a spatial frequency of the shadow pattern formed by the shaded regions 122 of the floaters (e.g., a two-dimensional Fourier transform of the shadow pattern), a spatial and temporal frequency of the shadow pattern (e.g., a three-dimensional Fourier transform of the shadow patterns of a series of video frames 120)

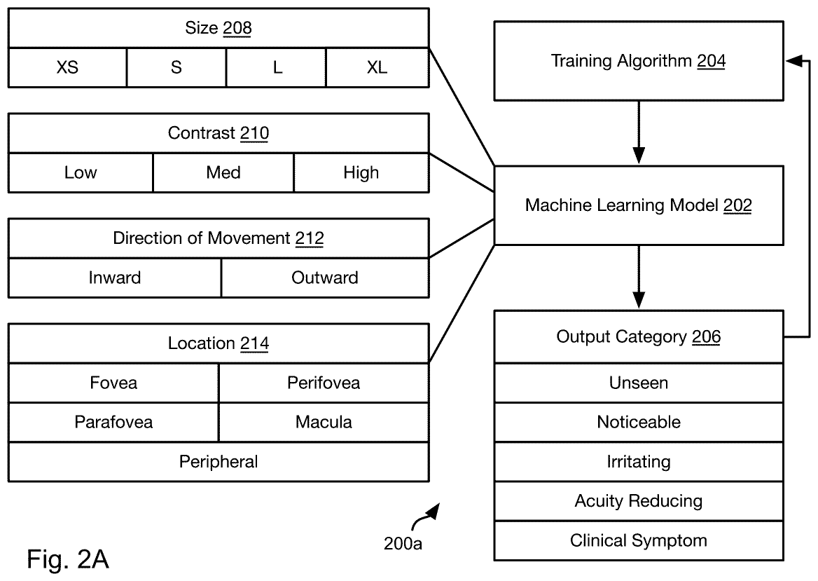

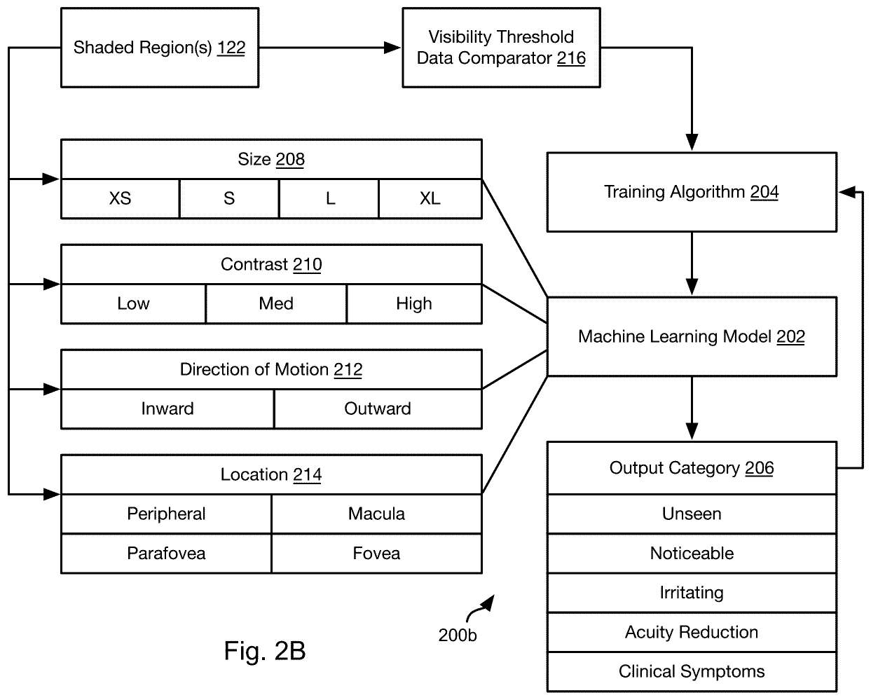

FIG. 2A illustrates a system 200a for training a machine learning model 202 to characterize the clinical significance of floaters 114 in an eye 100 based on one or more shaded regions 122 in one or more images 120 of the retina 112 of the eye 100. The machine learning model may be implemented as a neural network, deep neural network, convolution neural network, multiple linear regression model, random sample consensus regression model, multiple polynomial regression model, support vector regression model, Bayesian neural network, genetic algorithm, or any other type of machine learning model.

The machine learning model 202 may be trained by one or more training algorithms 204 to output a category 206 estimating the clinical significance of the floaters 114. The clinical significance may be one of a set of discrete set of values each corresponding to a degree of severity, such as unseen (e.g., not perceptible), noticeable, irritating, acuity reducing, or a clinical symptom (e.g., requiring treatment to maintain vision).

The machine learning model 202 may take as inputs metrics for a shaded region 122, such as the size 208, contrast 210, direction of movement 212, and location 214. These metrics may be obtained as described above with respect to FIG. 1B or by some other approach. Likewise, any of the other metrics described above with respect to FIG. 1B may be used in place of or in addition to those shown in FIG. 2A.

In some implementation the size of a shaded region 122 is assigned to one of a set of bins representing a range of possible sizes, referred to herein with the following exemplary identifiers: extra small (XS), small (S), large (L), and extra large (XL). The input to the machine learning model 202 may therefore be an identifier of a bin mapped to a size range including the size of a given shaded region 122 in an image 120. Any number of bins may be used and each bin may be assigned any range of sizes that is non-overlapping with respect to the range of sizes of another bin.

In a like manner, a range of possible contrasts may be divided into non-overlapping sub-ranges that are each assigned to a bin having a bin identifier, e.g., high, medium, and low. The contrast 210 input to the machine learning model 202 for a shaded region may therefore be an identifier of the bin having the assigned sub-range including the contrast of the shaded region 122.

The direction of movement 212 input to the machine learning model 202 may be one of two values, one indicating movement toward the fovea 128 and the other indicating movement away from the fovea. Other characterizations of movement of the shaded region 122 may be alternatively or additionally be input to the machine learning model 202.

The location 214 that is input to the machine learning model 202 may be an identifier of the closest region of the retina 112 that is overlapped by the shaded region 122. For example, the identifier may correspond to some or all of the fovea, perifovea, parafovea, macula, or peripheral region of the retina 112. Alternatively, the distance between the center of the fovea and the point on the shaded region 122 that is closest to the fovea may be used. An identifier for a bin mapped to a range of distances including the distance may also be used as the location 214 input to the machine learning model 202.

Training of the machine learning model 202 may be accomplished using a plurality of training data entries. Each training data entry may include a set of inputs including some or all of the metrics described herein such as a size 208, contrast 210, direction of movement 212, location 214 of a shaded region 122, or any of the other example metrics described herein. Each training data entry further includes an assigned category as a desired output. The assigned category may be one of the output categories 206 assigned to the training data entry by a human expert based on an observation of the image 120 including the shaded region 122 or based on an observation of the patient whose eye is represented in the image 120. The assigned category may also be assigned by the patient rating the severity of the floater 114 that created the shaded region 122.

Each training data entry may be processed by inputting the inputs to the machine learning model 202, receiving an estimated category, and comparing the estimated category to the assigned category of the training data entry. The training algorithm 204 may then update the machine learning model 202 according to any difference, or “loss,” between the estimated category and the assigned category of the training data entry. For example, each category may be assigned an integer value i such that the loss function evaluated by the training algorithm is, or is a function of, |i1−i0|, where i1 is the index of the assigned category and i0 is the index of the estimated category.

During utilization, an image 120 is received, any shaded regions 122 are identified and metrics thereof calculated. The metrics are processed using the machine learning model 202 to obtain an estimated category that is then output to a user, such as on a display device.

FIG. 2B illustrates an alternative system 200b for training a machine learning model to characterize the clinical significance of floaters using visibility threshold data. Visibility threshold data includes experimental data that characterizes the ability of a human observer to distinguish a feature. The experimental data may include data specific to floaters and may additionally or alternatively include data that is not specific to floaters, such as data used to characterize the effectiveness of camouflage. In particular, the experimental data may define a threshold surface with respect to three or two variables, the threshold surface defining the boundary between combinations of values for the two or more variables that are visible and those combinations of the two or more values that are not visible.

Non-limiting example sources of visibility threshold data may include any of the following, all of which are hereby incorporated herein by reference in their entirety: “Motion and Vision. II. Stabilized Spatio-Temporal Threshold Surface,” D. H. Kelly, J. Opt. Soc. Am., Vol. 69, No. 10 (October 1979); “Contrast Sensitivity of the Human Eye and its Effects on Image Quality,” Barten, P. G. (1999); “The contrast sensitivity gradient across the human visual field: With emphasis on the low spatial frequency range,” Pointer, J. S., & Hess, R. F. Vision Research, 29(9), 1133-1151 (1989); “Visual Processing of Moving Stimuli,” D. H. Kelly, J. Opt. Soc. Am., Vol. 2, No. 2 (February 1985); “Retinal Inhomogeneity. I. Spatiotemporal Contrast Sensitivity,” J. Opt. Sec. Am., Vol. 1, No. 1 (January 1984); “Retinal Inhomogeneity. II. Spatial Summation,” J. Opt. Soc. Am., Vol. 1, No. 1 (January 1984); “Retinal Inhomogeneity. III. Circular-Retina Theory,” D. H. Kelly, J. Opt. Soc. Am., Vol. 2, No. 6 (June 1985); “Motion and Vision. II. Stabilized Spatio-Temporal Threshold Surface,” J. Opt. Soc. Am., Vol. 69, No. 10 (October 1979); “Contrast Sensitivity of the Human Eye and Its Effects on Image Quality,” P. G. J. Barten, SPIE Optical Engineering Press (1999).

Non-limiting examples of variables that may define a threshold surface may include: spatial frequency (cycles per degree of field of view); temporal frequency (Hz); velocity (degrees per second); modulation (e.g., amplitude of spatial or temporal contrast); and eccentricity (e.g., distance from the fovea measured in degrees)

Examples of threshold surfaces include a first surface defined with respect to spatial frequency, velocity, and/or modulation amplitude and a second surface defined with respect to spatial frequency, eccentricity, and modulation amplitude; a third surface defined with respect to spatial frequency, temporal frequency, and/or modulation amplitude).

The system 200b may include a visibility threshold data comparator 216 that may evaluate a shadow pattern of one or more shaded regions 122 with respect to experimental data. For example, for each threshold surface of one or more threshold surfaces defined by two or more variables, the visibility threshold data comparator 216 may compute values for the two or more variables for the shadow pattern and evaluate the values with respect to the two or more variables to determine whether the values for the two or more variables indicate that the shadow pattern is visible.

The visibility threshold data comparator 216 may perform one or more evaluations with respect to each threshold surface. The one or more evaluations may include evaluating whether the values for the two or more variables for the shadow pattern are above the threshold surface, i.e., are visible. The one or more evaluations may include calculating a distance (D_{T}(j)) between the values for the two or more variables defining a threshold surface j and a closest point on the threshold surface j, where j is an index assigned to each threshold surface used.

The machine learning model 202 may take the same inputs as for the system 200a and may output an estimated category as for the system 200a. The training data entries used to train the machine learning model 202 may likewise be the same as described above with respect to the system 200a. However, the training algorithm 204 may be modified relative to that described with respect to the system 200a. More particularly, the training algorithm 204 may evaluate the estimated category obtained by processing the inputs of a training data entry with respect to the output of the visibility threshold data comparator 216. For example, let each possible output category 206 have an index i. Each image 120 of a set of images may be assigned an assigned category i by a human evaluator, such as a trained medical professional or the patient of whom each image 120 was taken. Values for the variables defining each threshold surface j may also be calculated for each image 120. The distance D_{T}(j) of the values of each image from each threshold surface j may be calculated. For each output category i and threshold surface j, there will therefore be a distribution of distances of images 120 assigned that output category i. This distribution may be used to define a probability distribution Pᵢⱼ(D_{T}(j)) such that for any given distance D_{T}(j), Pᵢⱼ(D_{T}(j)) is the probability that an image 120 assigned category i will be at distance D_{T}(j) from the threshold surface j.

Supposing that the estimated category for a given set of one or more input images 120 is i0. The loss function used by the training algorithm 204 may be calculated by calculating D_{T}(j) of the shadow pattern of the input image 120 and obtaining Pᵢ₀ⱼ (D_{T} (j)) with respect to each threshold surface j.

The loss function may be a function of Pᵢ₀ⱼ (D_{T} (j)) for all of the threshold surfaces j, such that the loss function increases as the values of Pᵢ₀ⱼ (D_{T} (j)) decrease. For example, the loss function may be, or be a function of, Σⱼ₌₁^{N}/Pᵢ₀ⱼ (D_{T} (j)) or Σⱼ₌₁^{N}1=Pᵢ₀ⱼ (D_{T} (j)), where N is the number of threshold surfaces. Note that the previous equations are exemplary only and other functions of Pᵢ₀ⱼ (D_{T} (j)) may also be used such that the loss function increases as the values of Pᵢ₀ⱼ (D_{T} (j)) decrease. The loss function may additionally be a function of a difference between the estimated category and assigned category of the training data entry. For example, the loss function may additionally be a function of, |i1−i0|, where it is the index of the assigned category and i0 is the index of the estimated category.

The training algorithm 204 may train the machine learning model 202 in order to minimize the loss function within any constraints (e.g., available data, number of iterations possible).

Using the above-described approach, the training algorithm 204 will train the machine learning model 202 to attempt to assign each image 120 to an output category 206 such that the distance D_{T}(j) of the image 120 is closer to the peak of the probability distribution of that category 206 than to the peaks of the probability distributions of the other possible categories 206. Since the probability distributions are more continuous relative to a finite number of categories (e.g., the illustrated five), the training algorithm 204 can more easily converge on suitable parameters for the machine learning model 202.

Upon training, utilization of the machine learning model 202 may be the same as described above with respect of the system 200a. In some embodiments, the visibility threshold data is not used during utilization.

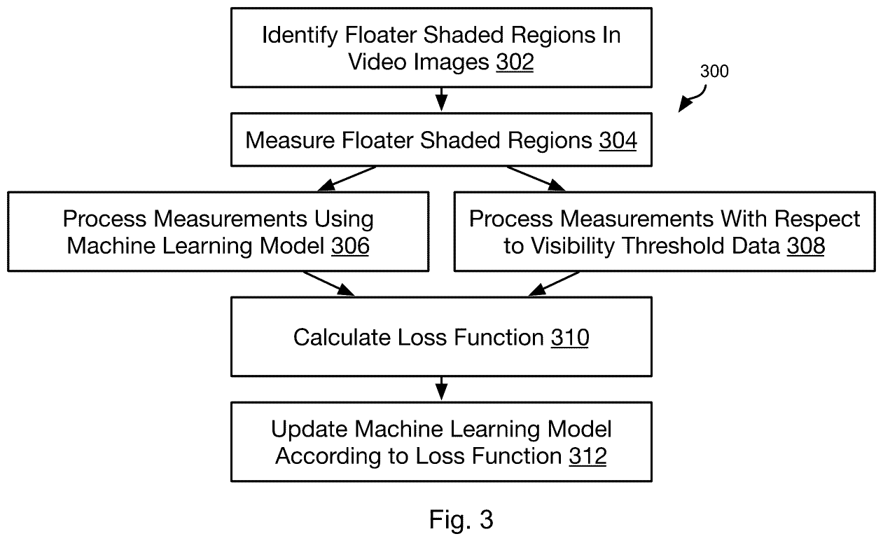

FIG. 3 illustrates a method 300 for training a machine learning model 202 to determine the clinical significance of floaters using visibility threshold data. The method 300 may be performed with respect to images 120. For example, for each of eye of each patient of a plurality of patients a series of two or more images 120 may be used to generate each training data entry. Each series of two or more images 120 may have a corresponding category 206 assigned by a human, such as the patient or a trained expert (“the assigned category”).

The method 300 may include identifying, at step 302, shaded regions 122 in the two or more images 120 that correspond to floaters 114. As noted above, shaded regions 122 may be identified based on movement indicated in the two or more images 120 or using any other approach.

The method 300 may include measuring, at step 304, the shaded regions 122. Measuring the shaded regions 122 may include obtaining any of the metrics described above with respect to FIG. 2A (size 208, contrast 210, direction of movement 212, location 214, etc.). Measuring the shaded regions may include calculating values for any of the above-described variables that are used to define any of the visibility threshold surfaces used in the method 300.

The method 300 may include processing, at step 306, some or all of the metrics described above with respect to FIG. 2A using the machine learning model 202 to obtain an estimated category. The method 300 further includes processing, at step 308, measurements of the shaded regions 122 to obtain values for two or more variables defining one or more visibility threshold surfaces.

The method 300 may include calculating, at step 310, a loss function with respect to the estimated category and the values for the two or more values. For example, the loss function may be a function of the two or more values and the probability distribution for the estimated category as described above. The machine learning model 202 is then updated at step 312 by the training algorithm 204.

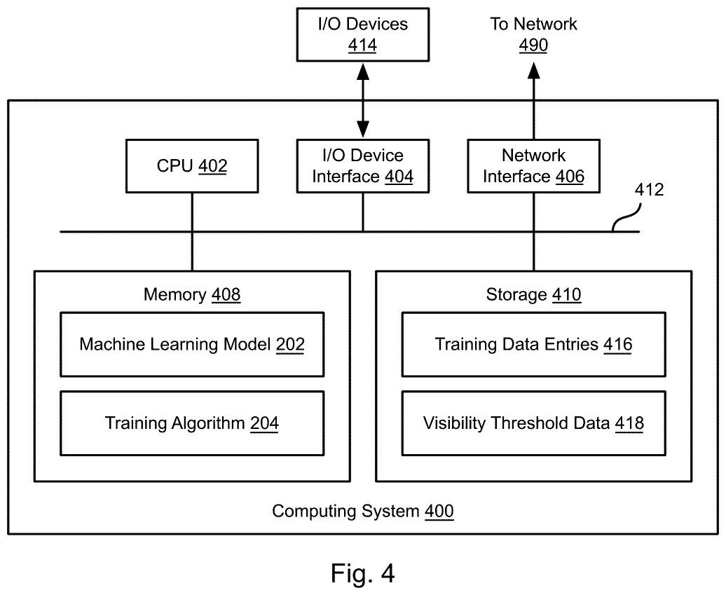

FIG. 4 illustrates an example computing system 400 that implements, at least partly, one or more functionalities described herein with respect to FIGS. 1A to 3. The computing system 400 may be integrated with an imaging device, such as an SLO, or be a separate computing device receiving images of a patient's eye from the imaging device.

As shown, computing system 400 includes a central processing unit (CPU) 402, one or more I/O device interfaces 404, which may allow for the connection of various I/O devices 414 (e.g., keyboards, displays, mouse devices, pen input, etc.) to computing system 400, network interface 406 through which computing system 400 is connected to network 490 (which may be a local network, an intranet, the internet, or any other group of computing systems communicatively connected to each other, as described in relation to FIG. 1), a memory 408, storage 410, and an interconnect 412.

In cases where computing system 400 is an imaging system, such as a digital microscope, OCT microscope, or SLO, computing system 400 may further include one or more optical components for obtaining ophthalmic imaging of a patient's eye as well as any other components known to one of ordinary skill in the art. In cases where computing system 400 is a surgical microscope, computing system 400 may further include many other components known to one of ordinary skill in the art to perform the ophthalmic surgeries described herein as known to one of ordinary skill in the art.

CPU 402 may retrieve and execute programming instructions stored in the memory 408. Similarly, CPU 402 may retrieve and store application data residing in the memory 408. The interconnect 412 transmits programming instructions and application data, among CPU 402, I/O device interface 404, network interface 406, memory 408, and storage 410. CPU 402 is included to be representative of a single CPU, multiple CPUs, a single CPU having multiple processing cores, and the like.

Memory 408 is representative of a volatile memory, such as a random access memory, and/or a nonvolatile memory, such as non-volatile random access memory, phase change random access memory, or the like. As shown, memory 408 may store the machine learning model 202 during training and utilization. For the computing system 400 used to train the machine learning model 202, the memory 408 may further store the training algorithm 204.

Storage 410 may be non-volatile memory, such as a disk drive, solid-state drive, or a collection of storage devices distributed across multiple storage systems. Storage 410 may optionally store training data entries 416 for training the machine learning model 202 using the approaches described above. The storage 410 may store the visibility threshold data 418, e.g., data describing the one or more visibility threshold surfaces and algorithms for calculating values for variables defining the one or more visibility threshold surfaces from a shadow pattern.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented, or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U. S. C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for characterizing floaters comprising:
receiving, by a computing device, one or more images of a patient's eye;
identifying, by the computing device, one or more shaded regions in the one or more images;
processing, by the computing device, the one or more shaded regions to obtain one or more measurements of the one or more shaded regions; and
processing, by the computing device, the one or more measurements using a machine learning model to obtain an estimated clinical significance of floaters in the patient's eye.

2. The method of claim 1, wherein the one or more measurements include a size of each of the one or more shaded regions.

3. The method of claim 1, wherein the one or more measurements include a contrast of each of the one or more shaded regions.

4. The method of claim 1, wherein the one or more images include a series of frames.

5. The method of claim 4, wherein the one or more measurements include a direction of movement of each of the one or more shaded regions.

6. The method of claim 5, wherein the direction of movement indicates only movement toward or away from a fovea of the patient's eye.

7. The method of claim 1, wherein the one or more measurements include a location of each of the one or more shaded regions relative to a fovea of the patient's eye.

8. The method of claim 1, wherein the one or more images include a series of video frames; and
wherein the one or more measurements include all of:
a size of each of the one or more shaded regions;
a contrast of each of the one or more shaded regions;
a direction of movement of each of the one or more shaded regions; and
a location of each of the one or more shaded regions relative to a representation of a fovea of the patient's eye in the one or more images.

9. The method of claim 1, wherein the one or more images include images from one of a scanning laser ophthalmoscope (SLO) and an optical coherence tomography (OCT) microscope.

10. The method of claim 1, wherein the estimated clinical significance of the floaters in the patient's eye comprises a selection from a set of possible output categories.

11. A method for training a machine learning model to characterize floaters comprising:
for each training data entry of a plurality of training data entries:
processing, by a computing device, input data from each training data entry with a machine learning model to obtain an estimated clinical significance of the floaters, the input data describing one or more shaded regions in one or more images of a retina of an eye of a patient;
(a) evaluating, by the computing device, the estimated clinical significance with respect to output data from each training data entry and visibility threshold data; and
updating, by the computing device, the machine learning model according to a result of (a).

12. The method of claim 11, wherein the one or more images include a series of video frames; and
wherein the input data includes one or more of:
a size of each of the one or more shaded regions;
a contrast of each of the one or more shaded regions;
a direction of movement of each of the one or more shaded regions; and
a location of each of the one or more shaded regions relative to a representation of a fovea of the eye of the patient in the one or more images.

13. The method of claim 11, wherein the visibility threshold data includes a visibility threshold surface defined by two or more variables.

14. The method of claim 13, wherein the two or more variables include two or more of:
spatial frequency;
temporal frequency;
modulation amplitude; and
eccentricity.

15. The method of claim 12, wherein the visibility threshold data includes a visibility threshold surface defined by two or more variables.

16. The method of claim 15, wherein the two or more variables include spatial frequency, velocity, and modulation amplitude.

17. The method of claim 15, wherein the two or more variables include spatial frequency, eccentricity, and modulation amplitude.

18. The method of claim 15, wherein the two or more variables include spatial frequency, temporal frequency, and modulation amplitude.

19. The method of claim 11, wherein the one or more images include images from a scanning laser ophthalmoscope.

20. The method of claim 11, wherein the estimated clinical significance comprises a selection from a set of possible output categories.
